(19) United States
(12) Patent Application Publication
(10) Pub. No.: US 2010/0016339 A1
(43) Pub. Date: Jan. 21, 2010

(54) FUNGICIDAL COMPOUNDS AND COMPOSITIONS

(75) Inventors: Jeremy Godwin, Stein (CH); Stephan Trah, Basel (CH); Clemens Lamberth, Basel (CH); Sebastian Volker Wendeborn, Basel (CH)

Correspondence Address: SYNGENTA CROP PROTECTION , INC.;PATENT AND TRADEMARK DEPARTMENT, 410 SWING ROAD, GREENSBORO, NC, 27409, US

(73) Assignee: SYNGENTA CROP PROTECTION, INC., Greensboro, NC (US)

(21) Appl. No.: 12/375,839

(22) PCT Filed: Jul. 30, 2007
(86) PCT No.: PCT/EP07/06720

§ 371 Date: Aug. 31, 2009

(30) Foreign Application Priority Data
Jul. 31, 2006 (GB) 0615213.6

(51) Int. Cl. A01N 43/90; C07D 237/02; A01N 43/58; A01P 3/00

(52) U.S. Cl. 514/262.1; 544/224; 514/247

(57) ABSTRACT

The present invention relates, inter alia, to a compound having the formula (I):—wherein (R)n is selected from the group consisting of 4-Br, 4-OCH₂CH₃, 4-OCH₂CF₃, 2-CH₃-4-Cl, 2-CH₃-4-OCH₃, 2-CH₃-4-OCF₃, 2-F-4-Br, 2-F-4-CF₃, 3-CH₃-4-Br, 3-F-4-Cl, 3-F-4-CH₃, 3-F-4-Br, 3-F-4-OCH₃ and 3-F-4-F. The present invention further relates to a fungicidal composition comprising a compound having the formula (I)—and also to fungicidal compositions comprising compounds having the formula (II) and/or (III): Furthermore, the present invention relates to methods for controlling pathogenic organisms using the compositions.

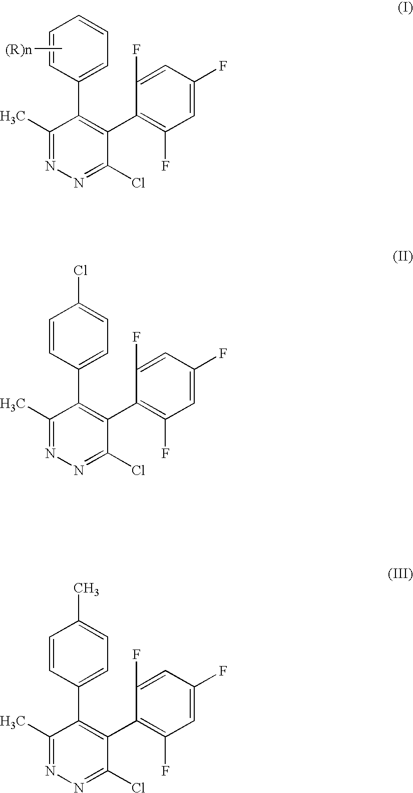

The present invention relates, inter alia, to fungicidal compounds and compositions for the treatment of phytopathogenic organisms, and to methods for controlling phytopathogenic organisms.

Crop yield can be adversely affected by disease. Therefore, the use of fungicides to control disease and/or reduce the effects of disease on crops is an important part of modern agricultural practice. Accordingly, there is a continuing need to provide improved fungicides and fungicidal compositions.

Thus, according to the present invention there is provided a compound having the formula (I):

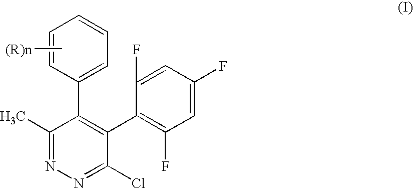

wherein (R)n is selected from the group consisting of 4-Br, 4-OCH₂CH₃, 4-OCH₂CF₃, 2-CH₃-4-Cl, 2-CH₃-4-OCH₃, 2-CH₃-4-OCF₃, 2-F-4-Br, 2-F-4-CF₃, 3-CH₃-4-Br, 3-F-4-Cl, 3-F-4-CH₃, 3-F-4-Br, 3-F-4-OCH₃ and 3-F-4-F.

The present invention further provides a fungicidal composition comprising a compound having the formula (I).

Further provided is a fungicidal composition comprising (i) a compound having the formula (I) and (ii) a fungicidal compound selected from the group consisting of cyproconazole, difenoconazole, epoxiconazole, fluquinconazole, flutriafol, ipconazole, metconazole, myclobutanil, penconazole, prothioconazole, prochloraz, propiconazole, tebuconazole, fenpropidine, fenpropimorph, spiroxamine, tridemorph, cyprodinil, fludioxonil, R-benalaxyl, R-metalaxyl, carbendazim, thiabendazole, iprodione, boscalid, penthiopyrad,

a compound having the formula F-1

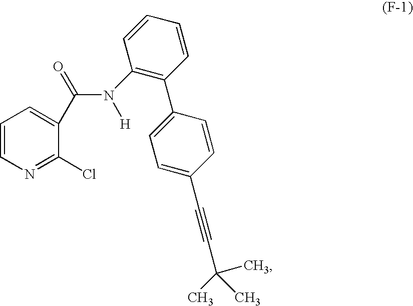

a compound having the formula F-2

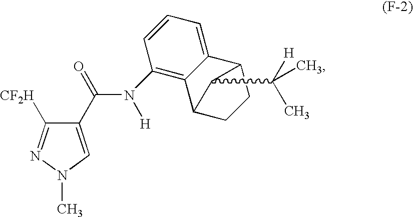

a compound having the formula F-3

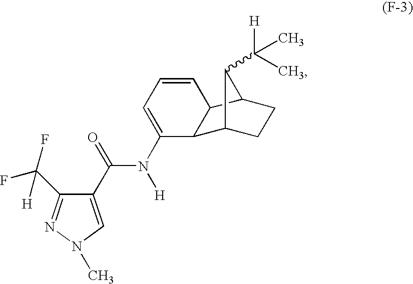

a compound having the formula F-4

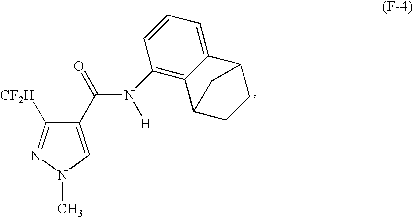

a compound having the formula F-5

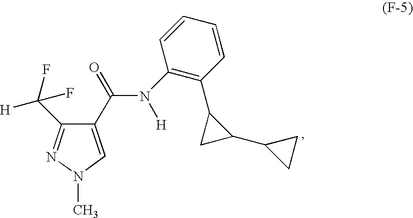

a compound having the formula F-6

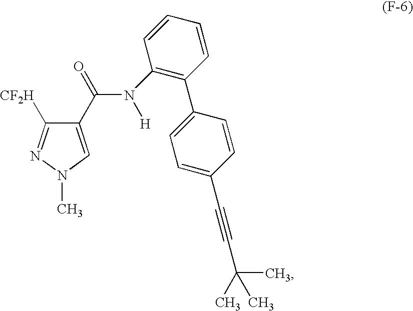

a compound having the formula F-7

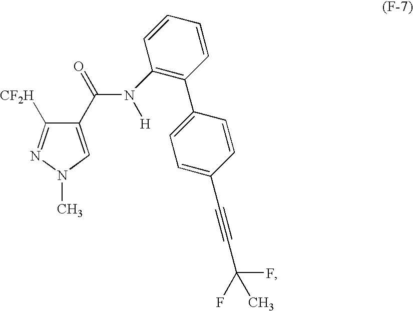

a compound having the formula F-8

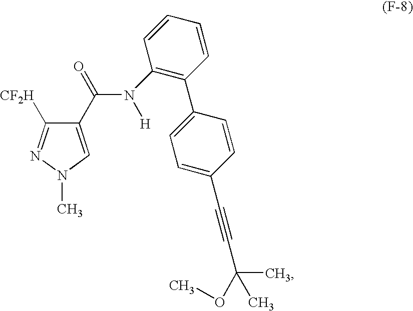

a compound having the formula F-9

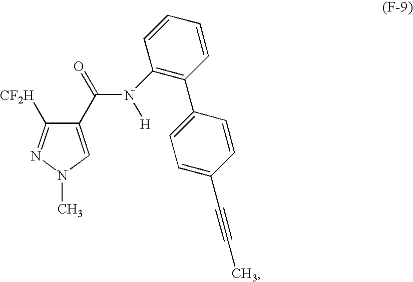

a compound having the formula F-10

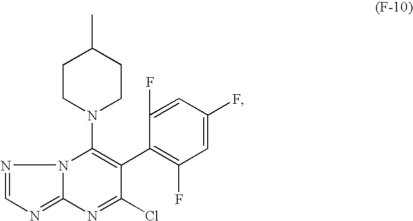

a compound having the formula F-11

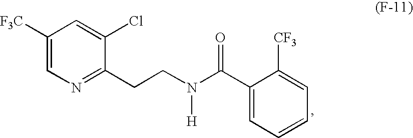

a compound having the formula F-12

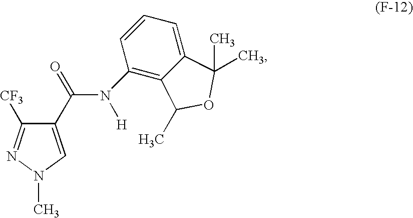

N-(3′,4′-dichloro-5-fluoro-1,1′-biphenyl-2-yl)-3-(difluoromethyl)-1-methyl-1H-pyrazole-4-carboxamide (compound F-13),
N-[2-(1,3-dimethylbutyl)-phenyl]-1,3-dimethyl-1H-pyrazole-4-carboxamide (compound F-14),
N-[2-(1,3-dimethylbutyl)-phenyl]-5-fluoro-1,3-dimethyl-1H-pyrazole-4-carboxamide (compound F-15),
N-[2-(1,3-dimethylbutyl)-phenyl]-5-chloro-1,3-dimethyl-1H-pyrazole-4-carboxamide (compound F-16),
3-(difluoromethyl)-N-[2-(1,3-dimethylbutyl)-phenyl]-1-methyl-1H-pyrazole-4-carboxamide (compound F-17),
3-(trifluoromethyl)-N-[2-(1,3-dimethylbutyl)-phenyl]-5-fluoro-1-methyl-1H-pyrazole-4-carboxamide (compound F-18),
3-(trifluoromethyl)-N-[2-(1,3-dimethylbutyl)-phenyl]-5-chloro-1-methyl-1H-pyrazole-4-carboxamide (compound F-19),
1,3-dimethyl-N-[2-(1,3,3-trimethylbutyl)-phenyl]-1H-pyrazole-4-carboxamide (compound F-20),
5-fluoro-1,3-dimethyl-N-[2-(1,3,3-trimethylbutyl)-phenyl]-1H-pyrazole-4-carboxamide (compound F-21),
3-(difluoromethyl)-1-methyl-N [2-(1,3,3-trimethylbutyl)-phenyl]-1H-pyrazole-4-carboxamide (compound F-22),
3-(trifluoromethyl)-1-methyl-N-[2-(1,3,3-trimethylbutyl)-phenyl]-1H-pyrazole-4-carboxamide (compound F-23),
3-(trifluoromethyl)-5-fluor-1-methyl-N-[2-(1,3,3-trimethylbutyl)-phenyl]-1H-pyrazole-4-carboxamide (compound F-24),
3-(trifluoromethyl)-5-chloro-1-methyl-N-[2-(1,3,3-trimethylbutyl)-phenyl]-1H-pyrazole-4-carboxamide (compound F-25),
3-(trifluoromethyl)-N-[2-(1,3-dimethylbutyl)-phenyl]-1-methyl-1H-pyrazole-4-carboxamide (compound F-26),
N-[2-(1,3-dimethylbutyl)-phenyl]-2-iodobenzamide (compound F-27),
2-iodo-N-[2-(1,3,3-trimethylbutyl)-phenyl]-benzamide (compound F-28),
N-[2-(1,3-dimethylbutyl)-phenyl]-2-(trifluoromethyl)-benzamide (compound F-29),
2-(trifluoromethyl)-N-[2-(1,3,3-trimethylbutyl)-phenyl]-benzamide (compound F-30),
3-difluoromethyl-1-methyl-1H-pyrazole-4-carboxylic acid N-[2-(1,1,2,2-tetrafluoroethoxy)phenyl]-amide (compound F-31),
3-difluoromethyl-1-methyl-1H-pyrazole-4-carboxylic acid N-[2-(1,1,2,3,3,3-hexafluoropropoxy)phenyl]-amide (compound F-32),
3-difluoromethyl-1-methyl-1H-pyrazole-4-carboxylic acid N-[2-(2-chloro-1,1,2-trifluoroethoxy)phenyl]-amide (compound F-33),
3-difluoromethyl-1-methyl-1H-pyrazole-4-carboxylic acid N-(4′-trifluoromethyl-biphen-2-yl)-amide (compound F-34),
3-difluoromethyl-1-methyl-1H-pyrazole-4-carboxylic acid N-(2′-trifluoromethyl-biphen-2-yl)-amide (compound F-35),
3-difluoromethyl-1-methyl-1H-pyrazole-4-carboxylic acid N-(2′-trifluoromethyl-biphen-2-yl)-amide (compound F-36),
   azoxystrobin, kresoxim-methyl, trifloxystrobin, orysastrobin, pyraclostrobin, mancozeb, captan, folpet, copper oxychloride, acibenzolar-S-methyl, benthiavalicarb, chlorothalonil, cymoxanil, dimethomorph, fluazinam, fluopicolide, iprovalicarb, cyazofamid, mandipropamid, metrafenone, pencycuron, pyroquilon and tricyclazole.

The present invention further provides a fungicidal composition comprising (i) a compound having the formula (II):

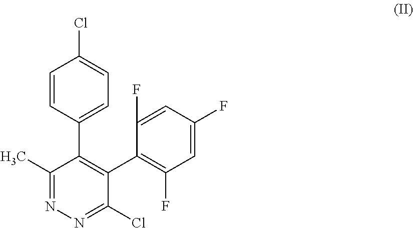

and (ii) a fungicidal compound selected from the group consisting of azoxystrobin, chlorothalonil, cyproconazole, epoxiconazole, prothioconazole, pyraclostrobin,
a compound having the formula F-3

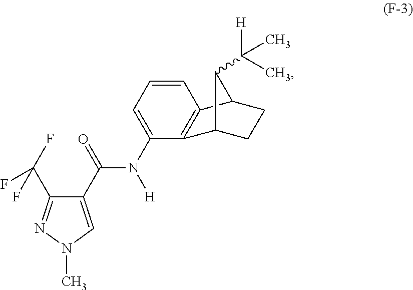

a compound having the formula F-4

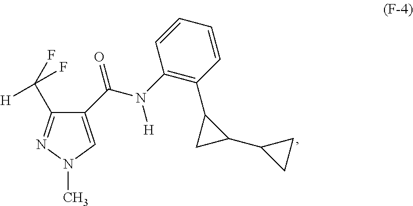

a compound having the formula F-9

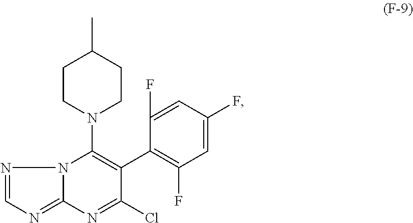

a compound having the formula F-10

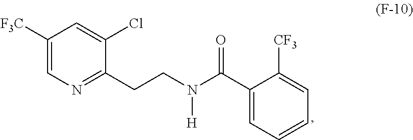

a compound having the formula F-37

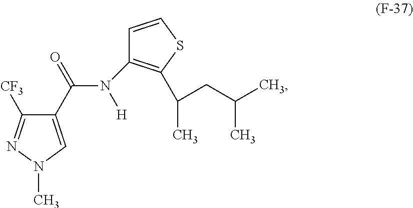

a compound having the formula F-38

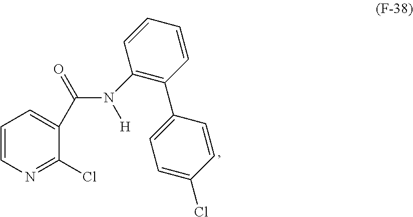

a compound having the formula F-39

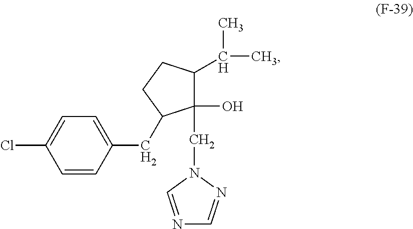

N-(3′,4′-dichloro-5-fluoro-1,1′-biphenyl-2-yl)-3-(difluoromethyl)-1-methyl-1H-pyrazole-4-carboxamide (compound F-13), and
a compound having the formula (I).

The present invention further provides a fungicidal composition comprising (i) a compound having the formula (III):

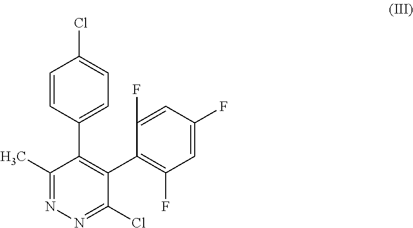

and (ii) a fungicidal compound selected from the group consisting of azoxystrobin, chlorothalonil, cyproconazole, epoxiconazole, prothioconazole, pyraclostrobin,
a compound having the formula F-3

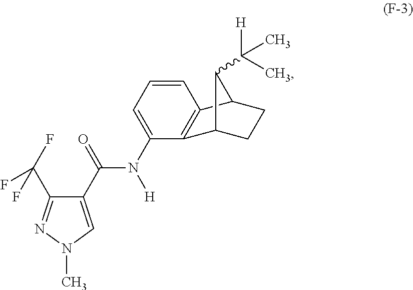

a compound having the formula F-4

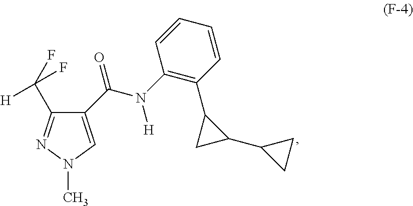

a compound having the formula F-9

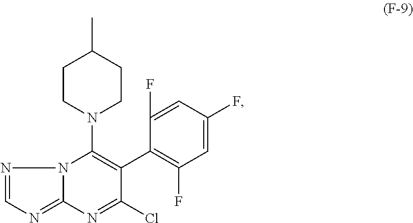

a compound having the formula F-10

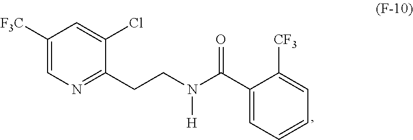

a compound having the formula F-37

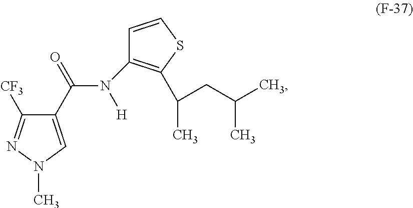

a compound having the formula F-38

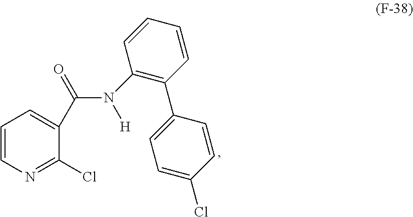

a compound having the formula F-39

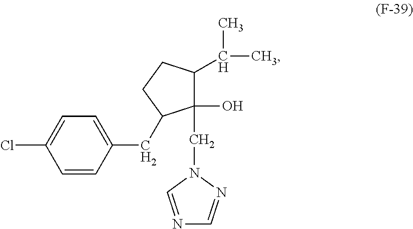

N-(3′,4′-dichloro-5-fluoro-1,1′-biphenyl-2-yl)-3-(difluoromethyl)-1-methyl-1H-pyrazole-4-carboxamide (compound F-13), and
a compound having the formula (I).

The present invention further provides a fungicidal composition comprising a compound having the formula (II) and a compound having the formula (III).

The fungicidal composition of the present invention may further comprise one or more agriculturally acceptable carriers and/or diluents.

The skilled person will appreciate that a number of the compounds referred to in this application can exist in different stereoisomeric forms and thus, for the avoidance of doubt, included in the present invention, where applicable, are compositions comprising different stereoisomeric forms. Where appropriate, the compounds referred to in this application may be provided in their free form or as an acceptable salt. Methods for making compounds referred to in this application are known in the art, for example in WO2005/121104, WO 2006/001175, WO 2004/016088, EP-0-545-099, EP-0-737-682, WO 04/058723, WO 2004/035589, WO 03/074491, WO 01/42223, U.S. Pat. No. 4,877,441, WO03/70705, WO 03/10149, WO 07/17450, WO 06/120219, U.S. Pat. No. 5,593,996, WO98/46607, EP-0-267-778, U.S. Pat. No. 4,938,792, EP-0-545-099, U.S. Pat. No. 5,589,493, EP-0-737-682, U.S. Pat. No. 5,869,517, U.S. Pat. No. 5,747,518 and WO 04/016088.

The compositions of the present invention can be used to control susceptible organisms that cause phytopathogenic diseases, in particular they can be used to control phytopathogenic fungi. Fungicidal compositions comprising two or more compounds may exhibit a synergistic effect. A synergistic effect exists whenever the action of an active ingredient combination is greater than the sum of the actions of the individual components.

A synergistic effect can be calculated using the so-called COLBY formula (COLBY, S. R. “Calculating synergistic and antagonistic responses of herbicide combination”. Weeds, Vol. 15, pages 20-22; 1967):

ppm=milligrams of active ingredient (=a.i.) per litre of spray mixture
X=% action by active ingredient A) using p ppm of active ingredient
Y=% action by active ingredient B) using q ppm of active ingredient.

According to COLBY, the expected (additive) action of active ingredients (A)+(B) using p+q ppm of active ingredient is

$$E = X + Y - \frac{X \cdot Y}{100}$$

If the action actually observed (O) is greater than the expected action (E), then the action of the combination is super-additive, i.e. there is a synergistic effect. In mathematical terms the synergism factor SF corresponds to O/E. In the agricultural practice an SF of ≧1.2 indicates significant improvement over the purely complementary addition of activities (expected activity), while an SF of ≦0.9 in the practical application routine signals a loss of activity compared to the expected activity.

Besides an improved or synergistic action, the fungicidal compositions according to the present invention may also have further surprising advantageous properties. Examples of such advantageous properties that may be mentioned are: a broadening of the spectrum of fungicidal activity to other phytopathogens, for example to resistant strains; a reduction in the rate of application of the active ingredients; more advantageous degradability; improved toxicological and/or ecotoxicological behaviour; or improved characteristics of the useful plants including: emergence, crop yields, more developed root system, tillering increase, increase in plant height, bigger leaf blade, less dead basal leaves, stronger tillers, greener leaf colour, less fertilizers needed, less seeds needed, more productive tillers, earlier flowering, early grain maturity, less plant verse (lodging), increased shoot growth, improved plant vigour, and early germination.

The compositions of the present invention may also comprise additional active ingredients such as a pesticide (e.g an insecticide, a nematicide, a miticide or bactericide), a (additional) fungicide, a herbicide, or a plant growth regulator where appropriate. An additional active ingredient may provide a composition having a broader spectrum of activity or increased persistence at a locus; further synergise the activity or complement the activity (for example by increasing the speed of effect) of the composition; or help to overcome or prevent the development of resistance to individual components. The particular additional active ingredient will depend upon the intended utility of the composition. Examples of suitable pesticides include the following:

Pyrethroids, such as permethrin, cypermethrin, fenvalerate, esfenvalerate, deltamethrin, cyhalothrin (in particular lambda-cyhalothrin), bifenthrin, fenpropathrin, cyfluthrin, tefluthrin, fish safe pyrethroids (for example ethofenprox), natural pyrethrin, tetramethrin, s-bioallethrin, fenfluthrin, prallethrin or 5-benzyl-3-furylmethyl-(E)-(1R,3S)-2,2-dimethyl-3-(2-oxothiolan-3-ylidenemethyl)cyclopropane carboxylate;
Organophosphates, such as, profenofos, sulprofos, acephate, methyl parathion, azinphos-methyl, demeton-s-methyl, heptenophos, thiometon, fenamiphos, monocrotophos, profenofos, triazophos, methamidophos, dimethoate, phosphamidon, malathion, chlorpyrifos, phosalone, terbufos, fensulfothion, fonofos, phorate, phoxim, pirimiphos-methyl, pirimiphos-ethyl, fenitrothion, fosthiazate or diazinon;
Carbamates (including aryl carbamates), such as pirimicarb, triazamate, cloethocarb, carbofuran, furathiocarb, ethiofencarb, aldicarb, thiofurox, carbosulfan, bendiocarb, fenobucarb, propoxur, methomyl or oxamyl;
Benzoyl ureas, such as diflubenzuron, triflumuron, hexaflumuron, flufenoxuron or chlorfluazuron;
Organic tin compounds, such as cyhexatin, fenbutatin oxide or azocyclotin;
Pyrazoles, such as tebufenpyrad and fenpyroximate;
Macrolides, such as avermectins or milbemycins, for example abamectin, emamectin benzoate, ivermectin, milbemycin, spinosad or azadirachtin;
Hormones or pheromones;
Organochlorine compounds such as endosulfan, benzene hexachloride, DDT, chlordane or dieldrin;
Amidines, such as chlordimeform or amitraz;
Fumigant agents, such as chloropicrin, dichloropropane, methyl bromide or metam;
Diacylhydrazines, such as tebufenozide, chromafenozide or methoxyfenozide;
Diphenyl ethers, such as diofenolan or pyriproxifen;

### Chlorfenapyr; Pymetrozine; Rynaxypyr or NNI-001;

Neonicotinoids such as acetamiprid, clothianidin, dinotefuran, imidacloprid, thiacloprid or thiamethoxam; or indoxacarb.

The compositions of the present invention may also include (additional) fungicides. Examples of such fungicides include:

Azoles, such as azaconazole, BAY 14120, bitertanol, bromuconazole, cyproconazole, difenoconazole, diniconazole, epoxiconazole, fenbuconazole, fluquinconazole, flusilazole, flutriafol, hexaconazole, imazalil, imibenconazole, ipconazole, metconazole, myclobutanil, pefurazoate, penconazole, prothioconazole, pyrifenox, prochloraz, propiconazole, simeconazole, tebuconazole, tetraconazole, triadimefon, triadimenol, triflumizole, triticonazole;
Pyrimidinyl carbinoles, such as ancymidol, fenarimol, nuarimol;
2-amino-pyrimidines, such as bupirimate, dimethirimol, ethirimol;
Morpholines, such as dodemorph, fenpropidine, fenpropimorph, spiroxamine, tridemorph;
Anilinopyrimidines, such as cyprodinil, mepanipyrim, pyrimethanil;
Pyrroles, such as fenpiclonil, fludioxonil;
Phenylamides, such as benalaxyl, furalaxyl, metalaxyl, R-metalaxyl, ofurace, oxadixyl;
Benzimidazoles, such as benomyl, carbendazim, debacarb, fuberidazole, thiabendazole;
Dicarboximides, such as chlozolinate, dichlozoline, iprodione, myclozoline, procymidone, vinclozoline;
Carboxamides, such as boscalid, carboxin, fenfuram, flutolanil, mepronil, oxycarboxin, penthiopyrad, thifluzamide;
Guanidines, such as guazatine, dodine, iminoctadine;
Strobilurines, such as azoxystrobin, dimoxystrobin, enestroburin, fluoxastrobin, kresoxim-methyl, metominostrobin, trifloxystrobin, orysastrobin, picoxystrobin, pyraclostrobin;
Dithiocarbamates, such as ferbam, mancozeb, maneb, metiram, propineb, thiram, zineb, ziram;
N-halomethylthiotetrahydrophthalimides, such as captafol, captan, dichlofluanid, fluoromides, folpet, tolyfluanid;
Copper-compounds, such as Bordeaux mixture, copper hydroxide, copper oxychloride, copper sulfate, cuprous oxide, mancopper, oxine-copper;
Nitrophenol-derivatives, such as dinocap, nitrothal-isopropyl;
Organo-phosphorus-derivatives, such as edifenphos, iprobenphos, isoprothiolane, phosdiphen, pyrazophos, tolclofos-methyl;
and various others, such as acibenzolar-S-methyl, anilazine, benthiavalicarb, blasticidin-S, chinomethionate, chloroneb, chlorothalonil, cyflufenamid, cymoxanil, dichlone, diclocymet, diclomezine, dicloran, diethofencarb, dimethomorph, flumorph, dithianon, ethaboxam, etridiazole, famoxadone, fenamidone, fenoxanil, fentin, ferimzone, fluazinam, fluopicolide, flusulfamide, fenhexamid, fosetyl-aluminium, hymexazol, iprovalicarb, cyazofamid, kasugamycin, mandipropamid, methasulfocarb, metrafenone, nicobifen, pencycuron, phthalide, polyoxins, probenazole, propamocarb, proquinazid, pyroquilon, quinoxyfen, quintozene, sulfur, tiadinil, triazoxide, tricyclazole, triforine, validamycin, or zoxamide.

The composition of the present invention may further comprise one or more herbicides—for example bromoxynil, an HPPD inhibitor (e.g mesotrione), an ACCase inhibitor, an ALS inhibitor (e.g primisulfuron, prosulfuron and trifloxysulfuron), an EPSPS (5-enol-pyrovyl-shikimate-3-phosphate-synthase) inhibitor (e.g glyphosate), a glutamine synthetase inhibitor (e.g glufosinate) and/or a PPO (protoporphyrinogen-oxidase) inhibitor.

The present invention further provides a pharmaceutical composition comprising a compound of formula (I) or a fungicidal composition of the present invention, and a pharmaceutically inert carrier or diluent.

The present invention still further provides a method for treating or preventing infection of a pathogenic organism in a human or animal comprising administering to the human or animal a pharmaceutical composition comprising a compound of formula (I) or a fungicidal composition of the present invention.

The present invention still further provides a pharmaceutical composition comprising a compound of formula (I) or a fungicidal composition of the present invention for therapeutic or prophylactic use.

The present invention further provides the use of a pharmaceutical composition comprising a compound of formula (I) or a fungicidal composition of the present invention, in the manufacture of a medicament for the treatment of a disease caused by a pathogenic organism, especially a fungus.

A further aspect of the present invention is a method of controlling a phytopathogenic organism which comprises applying to the organism, to a locus of the organism, to a plant, or to plant propagation material an effective amount of a fungicidal composition comprising a compound of formula (I) or a fungicidal composition of the present invention.

The compositions according to the invention are useful for controlling susceptible phytopathogenic fungi of the following classes: Fungi imperfecti (e.g. Botrytis spp., Alternaria spp.) and Basidiomycetes (e.g. Rhizoctonia spp., Hemileia spp., Puccinia spp., Phakopsora spp., Ustilago spp., Tilletia spp.). Additionally, they are also effective against Ascomycetes (e.g. Venturia spp., Blumeria spp., Podosphaera leucotricha, Monilinia spp., Fusarium spp., Uncinula spp., Mycosphaerella spp., Pyrenophora spp., Rhynchosporium secalis, Magnaporthe spp., Colletotrichum spp., Gaeumannomyces graminis, Tapesia spp., Ramularia spp., Microdochium nivale, Sclerotinia spp.) and Oomycetes (e.g. Phytophthora spp., Pythium spp., Plasmopara spp., Pseudoperonospora cubensis). Furthermore, the compositions of the present invention are also useful for controlling susceptible phytopathogenic bacteria and viruses (for example Xanthomonas spp, Pseudomonas spp and Erwinia amylovora).

The term “plant” as used herein includes, for example, the following species of plants: grape vines; cereals, such as wheat, barley, rye or oats; beet, such as sugar beet or fodder beet; fruits, such as pomes, stone fruits or soft fruits, for example apples, pears, plums, peaches, almonds, cherries, strawberries, raspberries or blackberries; leguminous plants, such as beans, lentils, peas or soybeans; oil plants, such as rape, mustard, poppy, olives, sunflowers, coconut, castor oil plants, cocoa beans or groundnuts; cucumber plants, such as marrows, cucumbers or melons; fibre plants, such as cotton, flax, hemp or jute; citrus fruit, such as oranges, lemons, grapefruit or mandarins; vegetables, such as spinach, lettuce, asparagus, cabbages, carrots, onions, tomatoes, potatoes, cucurbits or paprika; lauraceae, such as avocados, cinnamon or camphor; maize; tobacco; nuts; coffee; sugar cane; tea; vines; hops; durian; bananas; natural rubber plants; turf or ornamentals, such as flowers, shrubs, broad-leaved trees or evergreens, for example conifers. However, this list does not represent any limitation. The term “plant” also includes transgenic plants.

The term “locus” as used herein embraces, inter alia, the place in which the plant is growing or where the plant propagation material is sown, for example a field.

The term “plant propagation material” includes generative parts of a plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Also included are germinated plants and young plants which are to be transplanted after germination or after emergence from the soil. These young plants may be protected before transplantation by a total or partial treatment by immersion. The preferred “plant propagation material” is seeds.

The amount of a composition according to the invention to be applied will depend on various factors, such as the compounds employed; the subject of the treatment, such as, for example plants, soil or seeds; the type of treatment, such as, for example spraying, dusting or seed dressing; the purpose of the treatment, such as, for example prophylactic or therapeutic; the type of fungi to be controlled or the application time.

Where the composition comprises two or more fungicidal components the weight ratio of the components is selected so as to give the desired activity. For example, where the composition comprises two fungicidal components (A and B) the weight ratio of component (A) to component (B) can be from 1000:1 to 1:1000, preferably from 100:1 to 1:100, more preferably from 20:1 to 1:20, for example 1:8; 1:5; 1:4 and 1:2.

The method of the invention comprises applying to the plants, the locus thereof or propagation material thereof in admixture or separately, a composition according to the invention which can be applied as a foliar, soil and/or seed treatment. Furthermore, the compositions of the invention may be applied before or after infection of the plant, locus or propagation material by the phytopathogenic organism.

When applied to the plant any appropriate application rate can be used, for example from 1 to 5000 g a.i./ha, particularly 10 to 1000 g a.i./ha, e.g. 50, 62.5, 75, 100, 125 or 200 g a.i./ha.

In agricultural practice the application rates of the compositions according to the invention depend on the type of effect desired, and typically range from 20 to 4000 g of total composition per hectare.

When the compositions according to the invention are used for treating seed, rates of 0.001 to 50 g of ai per kg of seed, preferably from 0.01 to 10 g ai per kg of seed are generally sufficient.

The compositions of the present invention may be provided in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such compositions may be produced in conventional manner, e.g. by mixing the active ingredients with appropriate inert formulation adjuvants (diluents, solvents, fillers and optionally other formulating ingredients such as surfactants, biocides, anti-freeze, stickers, thickeners and compounds that provide adjuvancy effects). Also conventional slow release formulations may be employed where long lasting efficacy is intended. Particularly formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the condensation product of formaldehyde with naphthalene sulphonate, an alkylarylsulphonate, a lignin sulphonate, a fatty alkyl sulphate, and ethoxylated alkylphenol and an ethoxylated fatty alcohol.

A seed dressing formulation is applied in a manner known per se to the seeds employing the compositions according to the invention and a diluent in suitable seed dressing formulation form, e.g. as an aqueous suspension or in a dry powder form having good adherence to the seeds. Such seed dressing formulations are known in the art. Seed dressing formulations may contain the single active ingredients or the combination of active ingredients in encapsulated form, e.g. as slow release capsules or microcapsules.

In general, the formulations include from 0.01 to 90% by weight of active ingredient, from 0 to 20% agriculturally acceptable surfactant and 10 to 99.99% solid or liquid formulation inerts and adjuvant(s), the active ingredient(s), and optionally other active agents, particularly microbiocides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent. Whereas commercial products will preferably be formulated as concentrates, the end user will normally employ diluted formulations.

The compounds and compositions can also be used for preventing or controlling fungi in other areas, for example in the protection of materials (e.g building materials such as wall boards and paint), wood and wood related products, in food storage and in hygiene management.

The Examples which follow illustrate example compositions of the present invention. The term “active ingredient” as used below may include two or more active ingredients.

### FORMULATION EXAMPLES

| Wettable powders | % |
|---|---|
| Active ingredient | 25% |
| Sodium lignosulfonate | 5% |
| Sodium lauryl sulphate | 3% |
| Highly dispersed silicic acid | 5% |
| Kaolin | 62% |

The active ingredient is thoroughly mixed with the adjuvants and the mixture is thoroughly ground in a suitable mill, affording wettable powders that can be diluted with water to give suspensions of the desired concentration.

| Powders for dry seed treatment | % |
|---|---|
| Active ingredient | 25% |
| Light mineral oil | 5% |
| Highly dispersed silicic acid | 5% |
| Kaolin | 65% |

The active ingredient is thoroughly mixed with the adjuvants and the mixture is thoroughly ground in a suitable mill, affording powders that can be used directly for seed treatment.

| Emulsifiable concentrate | % |
|---|---|
| Active ingredient | 13% |
| Octylphenol polyethylene glycol ether | 3% |
| (4-5 mol of ethylene oxide) | |
| Castor oil polyglycol ether (35 mol of ethylene oxide) | 4% |
| Cyclohexanone | 30% |
| Xylene mixture | 50% |

Emulsions of any required dilution, which can be used in plant protection, can be obtained from this concentrate by dilution with water.

| Dusts | % |
|---|---|
| Active ingredient | 5% |
| Talcum | 95% |

Ready-for-use dusts are obtained by mixing the active ingredient with the carrier and grinding the mixture in a suitable mill. Such powders can also be used for dry dressings for seed.

| Extruded granules | % |
|---|---|
| Active Ingredient | 15% |
| Sodium lignosulfonate | 2% |
| Carboxymethylcellulose | 1% |
| Kaolin | 82% |

The active ingredient is mixed and ground with the adjuvants, and the mixture is moistened with water. The mixture is extruded and then dried in a stream of air.

| Coated granules | % |
|---|---|
| Active ingredient | 8% |
| Polyethylene glycol (mol. wt. 200) | 3% |
| Kaolin | 89% |

The finely ground active ingredient is uniformly applied, in a mixer, to the kaolin moistened with polyethylene glycol. Non-dusty coated granules are obtained in this manner.

| Suspension concentrate | % |
|---|---|
| Active ingredient | 40% |
| Propylene glycol | 10% |
| Nonylphenol polyethylene glycol ether (15 mol of ethylene oxide) | 6% |
| Sodium lignosulfonate | 10% |
| Carboxymethylcellulose | 1% |
| Silicone oil (in the form of a 75% emulsion in water) | 1% |
| Water | 32% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

| Flowable concentrate for seed treatment | % |
|---|---|
| Active ingredient | 40% |
| Propylene glycol | 5% |
| Copolymer butanol PO/EO | 2% |
| Tristyrenephenole with 10-20 moles EO | 2% |
| l,2-benzisothiazolin-3-one (in the form of a 20% | 0.5% |
| solution in water) | |
| Monoazo-pigment calcium salt | 5% |
| Silicone oil (in the form of a 75% emulsion in water) | 0.2% |
| Water | 45.3% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by phytopathogenic organisms, by spraying, pouring or immersion.

1. A compound having the formula (I): 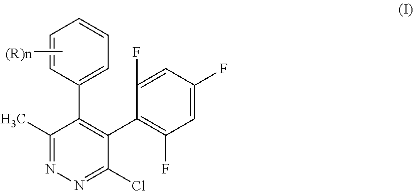
wherein (R)n is selected from the group consisting of 4-Br, 4-OCH₂CH₃, 4-OCH₂CF₃, 2-CH₃-4-Cl, 2-CH₃-4-OCH₃, 2-CH₃-4-OCF₃, 2-F-4-Br, 2-F-4-CF₃, 3-CH₃-4-Br, 3-F-4-Cl, 3-F-4-CH₃, 3-F-4-Br, 3-F-4-OCH₃ and 3-F-4-F.

2. A fungicidal composition comprising a compound according to claim 1.

3. A fungicidal composition comprising (i) a compound according to claim 1 and (ii) a fungicidal compound selected from the group consisting of cyproconazole, difenoconazole, epoxiconazole, fluquinconazole, flutriafol, ipconazole, metconazole, myclobutanil, penconazole, prothioconazole, prochloraz, propiconazole, tebuconazole, fenpropidine, fenpropimorph, spiroxamine, tridemorph, cyprodinil, fludioxonil, R-benalaxyl, R-metalaxyl, carbendazim, thiabendazole, iprodione, boscalid, penthiopyrad,
a compound having the formula F-1 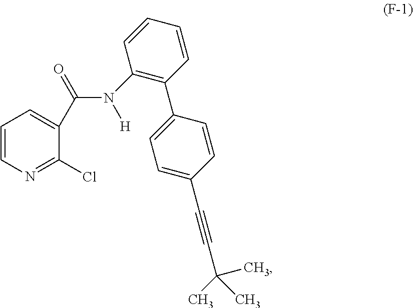
a compound having the formula F-2 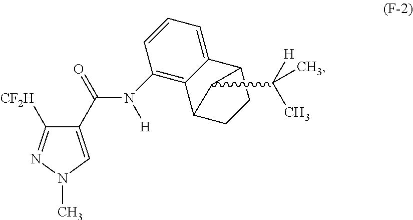
a compound having the formula F-3 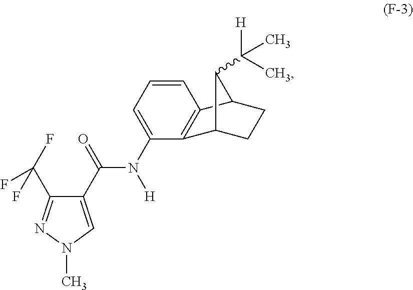
a compound having the formula F-4 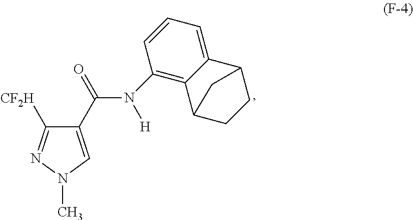
a compound having the formula F-5 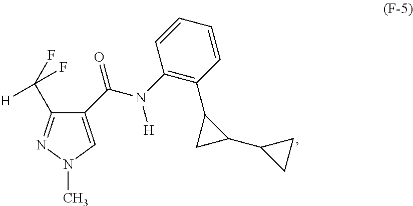
a compound having the formula F-6 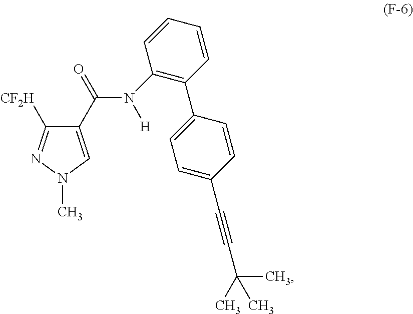
a compound having the formula F-7 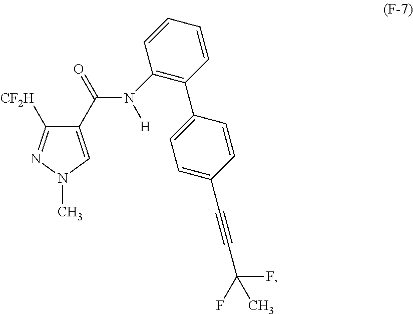
a compound having the formula F-8 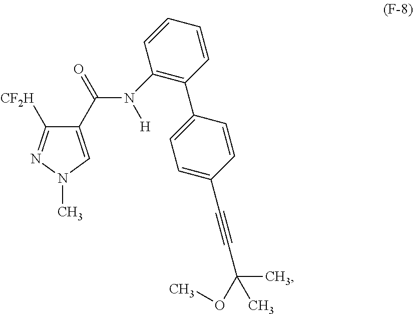
a compound having the formula F-9 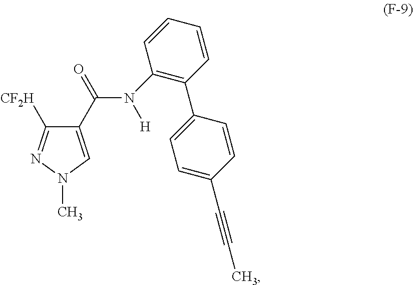
a compound having the formula F-10 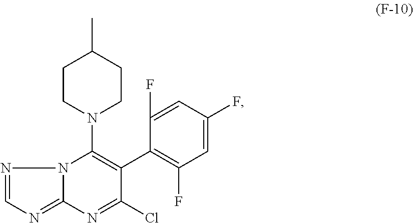
a compound having the formula F-11 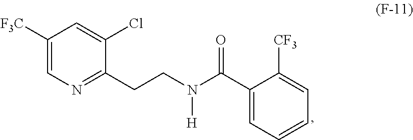
a compound having the formula F-12 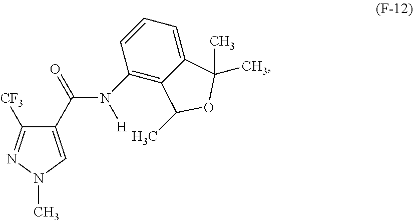
N-(3′,4′-dichloro-5-fluoro-1,1′-biphenyl-2-yl)-3-(difluoromethyl)-1-methyl-1H-pyrazole-4-carboxamide,
N-[2-(1,3-dimethylbutyl)-phenyl]-1,3-dimethyl-1H-pyrazole-4-carboxamide,
N-[2-(1,3-dimethylbutyl)-phenyl]-5-fluoro-1,3-dimethyl-1H-pyrazole-4-carboxamide,
N-[2-(1,3-dimethylbutyl)-phenyl]-5-chloro-1,3-dimethyl-1H-pyrazole-4-carboxamide,
3-(difluoromethyl)-N-[2-(1,3-dimethylbutyl)-phenyl]-1-methyl-1H-pyrazole-4-carboxamide,
3-(trifluoromethyl)-N-[2-(1,3-dimethylbutyl)-phenyl]-5-fluoro-1-methyl-1H-pyrazole-4-carboxamide,
3-(trifluoromethyl)-N-[2-(1,3-dimethylbutyl)-phenyl]-5-chloro-1-methyl-1H-pyrazole-4-carboxamide,
1,3-dimethyl-N-[2-(1,3,3-trimethylbutyl)-phenyl]-1H-pyrazole-4-carboxamide,
5-fluoro-1,3-dimethyl-N-[2-(1,3,3-trimethylbutyl)-phenyl]-1H-pyrazole-4-carboxamide,
3-(difluoromethyl)-1-methyl-N [2-(1,3,3-trimethylbutyl)-phenyl]-1H-pyrazole-4-carboxamide,
3-(trifluoromethyl)-1-methyl-N-[2-(1,3,3-trimethylbutyl)-phenyl]-1H-pyrazole-4-carboxamide,
3-(trifluoromethyl)-5-fluor-1-methyl-N-[2-(1,3,3-trimethylbutyl)-phenyl]-1H-pyrazole-4-carboxamide,
3-(trifluoromethyl)-5-chloro-1-methyl-N-[2-(1,3,3-trimethylbutyl)-phenyl]-1H-pyrazole-4-carboxamide,
3-(trifluoromethyl)-N-[2-(1,3-dimethylbutyl)-phenyl]-1-methyl-1H-pyrazole-4-carboxamide,
N-[2-(1,3-dimethylbutyl)-phenyl]-2-iodobenzamide,
2-iodo-N-[2-(1,3,3-trimethylbutyl)-phenyl]-benzamide,
N-[2-(1,3-dimethylbutyl)-phenyl]-2-(trifluoromethyl)-benzamide,
2-(trifluoromethyl)-N-[2-(1,3,3-trimethylbutyl)-phenyl]-benzamide,
3-difluoromethyl-1-methyl-1H-pyrazole-4-carboxylic acid N-[2-(1,1,2,2-tetrafluoroethoxy)phenyl]-amide,
3-difluoromethyl-1-methyl-1H-pyrazole-4-carboxylic acid N-[2-(1,1,2,3,3,3-hexafluoropropoxy)phenyl]-amide,
3-difluoromethyl-1-methyl-1H-pyrazole-4-carboxylic acid N-[2-(2-chloro-1,1,2-trifluoroethoxy)phenyl]-amide,
3-difluoromethyl-1-methyl-1H-pyrazole-4-carboxylic acid N-(4′-trifluoromethyl-biphen-2-yl)-amide,
3-difluoromethyl-1-methyl-1H-pyrazole-4-carboxylic acid N-(2′-trifluoromethyl-biphen-2-yl)-amide,
3-difluoromethyl-1-methyl-1H-pyrazole-4-carboxylic acid N-(2′-trifluoromethyl-biphen-2-yl)-amide,
azoxystrobin, kresoxim-methyl, trifloxystrobin, orysastrobin, pyraclostrobin, mancozeb, captan, folpet, copper oxychloride, acibenzolar-S-methyl, benthiavalicarb, chlorothalonil, cymoxanil, dimethomorph, fluazinam, fluopicolide, iprovalicarb, cyazofamid, mandipropamid, metrafenone, pencycuron, pyroquilon and tricyclazole.

4. A fungicidal composition comprising (i) a compound having the formula (II): 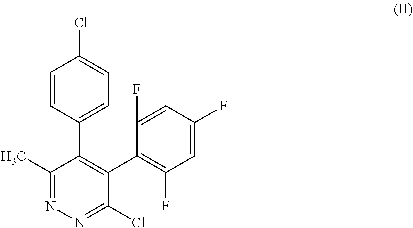
and (ii) a fungicidal compound selected from the group consisting of azoxystrobin, chlorothalonil, cyproconazole, epoxiconazole, prothioconazole, pyraclostrobin, a compound having the formula F-3 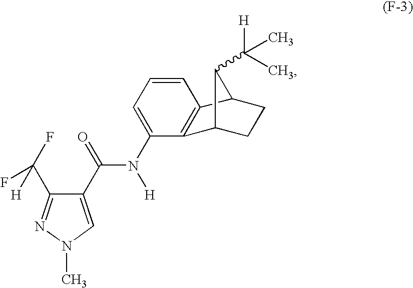
a compound having the formula F-4 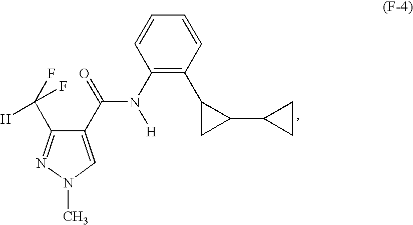
a compound having the formula F-9 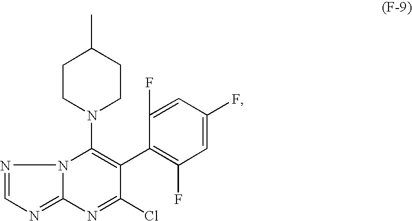
a compound having the formula F-10 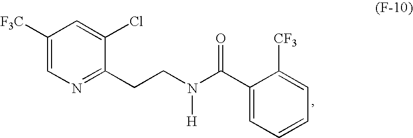
a compound having the formula F-37 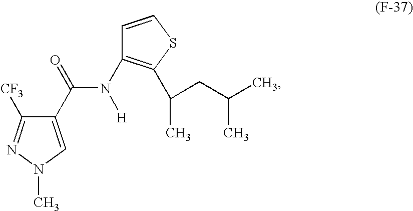
a compound having the formula F-38 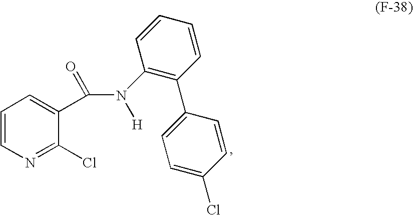
a compound having the formula F-39 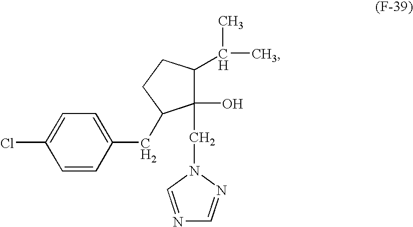
N-(3′,4′-dichloro-5-fluoro-1,1′-biphenyl-2-yl)-3-(difluoromethyl)-1-methyl-1H-pyrazole-4-carboxamide, and
a compound having the formula (I).

5. A fungicidal composition comprising (i) a compound having the formula (III): 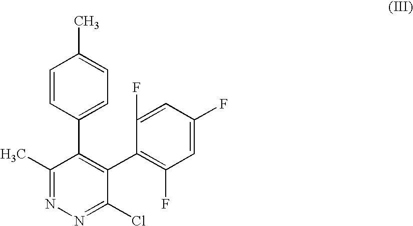
and (ii) a fungicidal compound selected from the group consisting of azoxystrobin, chlorothalonil, cyproconazole, epoxiconazole, prothioconazole, pyraclostrobin,
a compound having the formula F-3 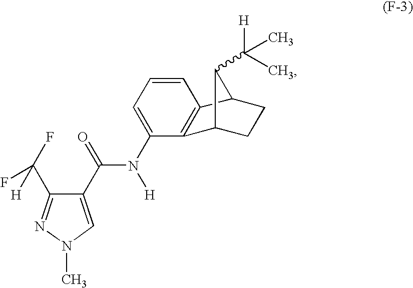
a compound having the formula F-4 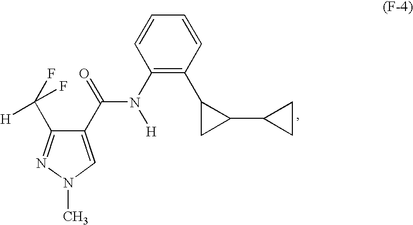
a compound having the formula F-9 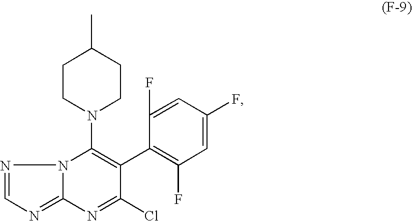
a compound having the formula F-10 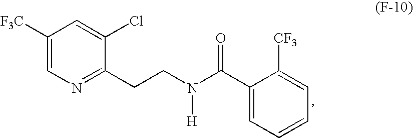
a compound having the formula F-37 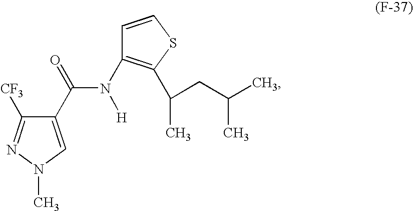
a compound having the formula F-38 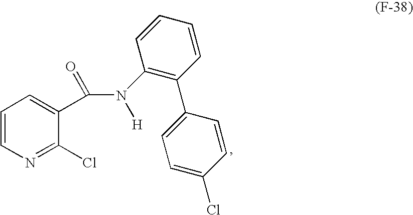
a compound having the formula F-39 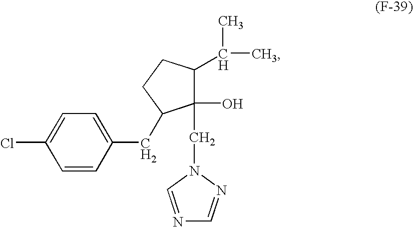
N-(3′,4′-dichloro-5-fluoro-1,1′-biphenyl-2-yl)-3-(difluoromethyl)-1-methyl-1H-pyrazole-4-carboxamide, and
a compound having the formula (I).

6. A fungicidal composition according to claim 3, wherein the ratio of (i) to (ii) is from 100:1 to 1:100.

7. A fungicidal composition according to claim 1, further comprising an agriculturally acceptable carrier or diluent.

8. A fungicidal composition according to claim 1, further comprising one or more additional active ingredient(s) selected from the group consisting of a herbicide, an insecticide, a nematicide, a miticide and a bactericide.

9. A pharmaceutical composition comprising a fungicidal compound according to claim 1.

10. A method of controlling a phytopathogenic organism which comprises applying to the organism, to a locus of the organism, to a plant, or to plant propagation material an effective amount of a fungicidal composition according to claim 2.

11. A method according to claim 10, wherein the phytopathogenic organism is a phytopathogenic fungus.

12. A method for treating or preventing infection of a pathogenic organism in a human or animal comprising administering to the human or animal a pharmaceutical composition according to claim 9.

13. (canceled)

14. (canceled)
